(19) United States
(12) Patent Application Publication
(10) Pub. No.: US 2020/0037950 A1
(43) Pub. Date: Feb. 6, 2020

(54) METHODS FOR BIOMECHANICAL MAPPING OF THE FEMALE PELVIC FLOOR

(71) Applicant: Advanced Tactile Imaging Inc., Trenton, NJ (US)

(72) Inventor: Vladimir Egorov, Princeton, NJ (US)

(21) Appl. No.: 16/055,265

(22) Filed: Aug. 6, 2018

(51) Int. Cl. A61B 5/00

(52) U.S. Cl. CPC A61B 5/4337; A61B 8/0858

(57) ABSTRACT

Methods for biomechanical mapping of the female pelvic floor may include the steps of inserting a vaginal tactile imaging probe into vagina; recording tactile responses for vaginal walls during vaginal wall deformation by moving a probe as well as dynamic pressure patterns during voluntary or involuntary muscle contractions in multiple test procedures; followed by calculating multiple biomechanical parameters characterizing vaginal tissue elasticity, pelvic support structures and dynamic pelvic functions. Individual biomechanical parameters may be visually represented by positioning their value within the established physiological parameter ranges varying from normal to diseased conditions. The methods may be used for identification of pelvic floor tissues with low elasticity, deteriorated or damaged pelvic support muscles and ligaments, and muscles with low contractive capability. Other methods include the steps of collecting clinical history and completing gynecological examinations of the pelvic floor and calculating probabilities of treatment success for pelvic diseases depending on a proposed treatment using a predictive mathematical model.

### GOVERNMENT-SPONSORED RESEARCH

This invention was made with the US Government support under grant No-SB1AG034714 awarded by the National Institute on Aging, National Institutes of Health. The Government has certain rights in this invention.

### BACKGROUND OF THE INVENTION

The present invention generally relates to characterization methods for female pelvic tissues. Specifically, the invention describes methods and devices for characterizing vaginal tissue elasticity, pelvic floor support structures and dynamic pelvic functions.

Recent survey identified as the highest priority research questions pertaining to pathophysiology and treatments of pelvic organ prolapse (POP). According to the survey, mechanistic research on pelvic supportive structures, clinical trials to optimize outcomes after POP surgery and evidence-based quality measures for POP outcomes are among the major focus areas [Siddiqui N Y, Gregory W T, Handa V L, DeLancey J O L, Richter H E, Moalli P, Barber M D, Pulliam S, Visco A G, Alperin M, Medina C, Fraser M O, Bradley C S. American Urogynecologic Society Prolapse Consensus Conference Summary Report. Female Pelvic Med Reconstr Surg. 2018 Jan. 5. PMID: 29309287]. In vaginal prolapse surgery, about 20% of procedures are performed for recurrent POP. Not many other fields of medicine have such poor surgical outcomes [Jeffery S, Roovers J P. Quo vadis, vaginal mesh in pelvic organ prolapse? International Urogynecology Journal 2018: 29: 1073-1074].

Changes in the elasticity of the vaginal walls, connective support tissues, and muscles are significant factors in the development of POP and stress urinary incontinence (SUI). Their high incidence dictates the need to develop new effective methods of objective pelvic tissue characterization and early disease detection [Egorov V, van Raalte H, Lucente V, Sarvazyan A. Biomechanical characterization of the pelvic floor using tactile imaging. In: Biomechanics of the Female Pelvic Floor, Eds. Hoyte L, Damaser M S, 1st Edition, Elsevier, 2016: 317-348.].

Traditionally, clinical diagnosis of POP, SUI, vaginal tissue atrophy and pelvic pain involves evaluating a medical history of a patient and performing a manual physical examination. During such manual examination a physician typically inspects the urogenital areas and rectum for masses and indication of reduced muscle tone. The physician then instructs the patient to cough, bear down or perform a Valsalva maneuver (a forceful attempt at exhalation with the mouth and nose closed) to see if and how far the vagina descends as the result of the additional abdominal pressure [Shagam J Y. Pelvic organ prolapse. Radiol Technol. 2006; 77(5):389-400].

While physical examination helps the clinician to describe the extent of pelvic disease conditions, it does not help in discerning the initial stage of abnormality development from the normal conditions or specifically identify the defective structures (muscles, ligaments) in the pelvic floor. Digital palpation does not provide quantitative tissue characterization to compare with normal elasticity of vaginal walls. It has poor sensitivity and is highly subjective.

Vaginal Tactile Imager (VTI) is an instrument intended to aid in diagnosis and objective evaluation of vaginal and pelvic floor conditions—as described in U.S. Pat. No. 8,840,571 and related patents. It allows objective assessment of tissue elasticity, pelvic floor support and function. However, the VTI cannot provide a comprehensive set of biomechanical parameters for characterization of female pelvic floor.

A need therefore exists for a method of creating a comprehensive biomechanical mapping of the pelvic floor tissues using an extended set of predetermined parameters, which may open a new possibility in biomechanical assessment and monitoring of pelvic floor conditions.

### SUMMARY OF THE INVENTION

The object of the present invention is to overcome the drawbacks of the prior art and to provide novel methods for biomechanical mapping of the female pelvic floor with a plurality of biomechanical parameters to characterize the biomechanical conditions of specific pelvic floor structures and functions.

A further object of the invention is to identify an extended set of parameters that can be obtained using a Vaginal Tactile Imager (VTI) in order to comprehensively characterize the pelvic floor tissues, pelvic support structures and pelvic functions contributing to POP development, and to establish their physiologic ranges—to allow visualization of some or all of these biomechanical parameters when acquired for a particular patient.

Another object of the invention is to provide methods for objective detection of pelvic organ disease or abnormality.

Yet another object of the invention is to provide methods for objective quantitative characterization of outcome of a surgical or another treatment of a pelvic floor disease.

In embodiments, a method for biomechanical mapping of the female pelvic floor may comprise the steps of:
(a) inserting a vaginal tactile imaging probe into vagina;
(b) recording tactile responses for vaginal walls during vaginal wall deformation and dynamic pressure patterns during muscle contractions during one or multiple test procedures;
(c) calculating multiple biomechanical parameters characterizing vaginal tissue elasticity, pelvic support structures and dynamic pelvic functions;
(d) representing one, some or all of the biomechanical parameters visually within their respective physiological parameter ranges varying from normal to diseased conditions; and
(e) identification of pelvic floor tissues with low elasticity, deteriorated or damaged pelvic support muscles and ligaments, and pelvic muscles with low contractive capability.

Step (b) of the method may further include recording tactile responses to:
low vaginal wall deformation caused by movement of a tactile probe by about 3-15 mm,
high vaginal wall deformation caused by movement of the tactile probe by about 15-45 mm,
no movement of the vaginal probe while performing voluntary and involuntary muscle contractions and relaxations by the patient.

Step (c) of the method may comprise calculating at least one biomechanical parameter for characterizing each of the three of the above circumstances.

In other embodiments, a method for biomechanical mapping of the female pelvic floor may comprise the steps (a) through (c) as listed above followed by the steps of:
(d) collecting clinical history and completing gynecological examinations of the pelvic floor; and
(e) calculating probabilities of treatment success for a particular pelvic disease condition for a variety of proposed treatments.

### BRIEF DESCRIPTION OF DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates transvaginal probe location after its insertion during recording of a tactile response pattern from two opposing vaginal walls—anterior and posterior compartments;

FIG. 2 shows anatomical locations for collecting tactile response data used for calculation of biomechanical parameters within the female pelvic floor;

FIG. 3 shows a correlation of various tactile response tests and biomechanical parameters calculated using data obtained during these tests, specific parameters are described in the text of the specification;

FIG. 4 is a flow chart illustrating a method for biomechanical mapping of the female pelvic floor;

FIG. 5 presents an example of visual presentation of biomechanical parameters as an indicator panel; and

FIG. 6 is a flow chart illustrating another method for biomechanical mapping of the female pelvic floor.

### DETAILED DESCRIPTION OF PREFERRED EMBODIMENTS OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

Specific terms are used in the following description, which are defined as follows: “tactile sensor” is the sensor configured to measure an applied orthogonal force averaged per sensor area or pressure. “Tissue deformation” is used to describe vaginal wall and adjacent structures deformation generally in orthogonal direction away from a vaginal canal—as caused by a movement of the tactile probe.

“Tactile Imaging” is a medical imaging modality translating the sense of touch into a digital image. The tactile image is a function of P (x, y, z), where P is the pressure on soft tissue surface under applied deformation and x, y and z are the coordinates where P was measured. The tactile image is a pressure map on which the direction of tissue deformation must be specified.

“Functional Tactile Imaging” translates muscle activity into dynamic pressure pattern P (x, y, t) for an area of interest, where t is time and x and y are coordinates where pressure P was measured. It may include: (a) muscle voluntary contraction, (b) involuntary reflex contraction, (c) involuntary relaxation, and (d) specific maneuvers.

“Biomechanical Mapping” is used herein to describe a combination of “Tactile Imaging” plus “Functional Tactile Imaging”.

A tactile imaging probe may be equipped with a pressure sensor array mounted on its external surface that acts similar to human fingers during a clinical examination. Movements of the probe may be used for deforming the soft tissue under examination and detecting the resulting changes in the pressure pattern on the probe's surface. The sensor head may be moved over the surface of the tissue to be studied, and the pressure response is evaluated at multiple locations along the tissue. The results and tactile response data may be used to generate 2D/3D images showing pressure distribution over the area of the tissue under study.

Generally, an inverse problem solution for tactile image P (x, y, z) would allow the reconstruction of tissue elasticity distribution (E) as a function of the same coordinates E (x, y, z). Unfortunately, the inverse problem solution is hardly possible for most real objects because it is a non-linear and ill-posed problem. However, the tactile image P (x, y, z) per se reveals tissue or organ anatomy and elasticity distribution because it maintains the stress-strain relationship for deformed tissue. Thus, the spatial gradients ∂P (x, y, z)/∂x, ∂p (x, y, z)/∂y, and ∂p (x, y, z)/∂z can be used in practice for soft tissue elasticity mapping, despite structural and anatomical variations.

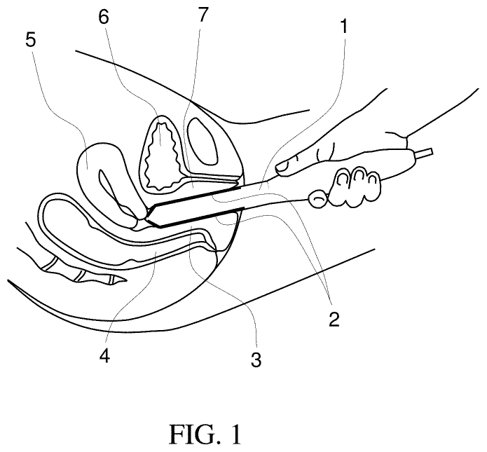

FIG. 1 illustrates a vaginal tactile imaging probe 1 positioned in vagina for recording of a pressure/tactile patterns by tactile sensor arrays 2 from two opposing vaginal walls. The tactile patterns may be recorded for the anterior 7 and the opposing posterior 3 vaginal compartments as well as for left and right sides of the vagina. Uterus 5 may be used as a reference point in presenting and analyzing tactile responses to vaginal tissue deformations and dynamic pressure patterns during pelvic muscle contraction. Rectum 4 and bladder 6 are shown as anatomical landmarks.

The vaginal tactile imaging probe 1, as shown in FIG. 1, may be equipped with a plurality of pressure (tactile) sensors spaced at 2.5 mm consecutively on both sides of the probe (96 individual sensors in one example), an orientation sensor, and a temperature controller configured to bring the probe temperature close to a human body before examination. The tactile imaging data may be sampled from the probe sensors and displayed on the Vaginal Tactile Imager (VTI) monitor in real time. The resulting pressure maps (tactile images) of the vagina integrate all the acquired pressure and positioning data for each of the pressure sensing elements. Additionally, the VTI may record the dynamic contraction of pelvic floor muscles with sufficient resolution, for example a resolution of at least 1 mm. A lubricating jelly may be used for patient comfort and to provide reproducible boundary/contact conditions with deformed tissues.

A VTI probe may be sized to cause 3-15 mm of tissue displacement and deformation, for example resulting from the probe initial insertion (Test 1). The probe may be subsequently moved to cause 20-45 mm of tissue deformation from the probe elevation (Test 2), and 5-7 mm of tissue deformation resulting from probe rotation (Test 3). The probe may also be used for recording of dynamic tactile responses during pelvic muscle contractions (Tests 4-8). The probe maneuvers in Tests 1-3 allow accumulation of multiple pressure patterns from the tissue surface to compose an integrated tactile image for the investigated area using probe orientation data. The spatial gradients ∂P(x, y)/∂y for anterior and posterior compartments may be calculated within the acquired tactile images in Tests 1 and 2; y-coordinate may be directed orthogonally from the longitudinal axis of the vaginal canal, x-coordinate may be located along the vaginal canal. The VTI probe may be equipped with a microprocessor containing software including data recording and analysis tools and reporting functions. It may be configured to present a visual representation of the anatomy, tactile pressure maps, and calculate (automatically) a plurality of predetermined parameters for at least some or all of the test procedures.

In embodiments, the pelvic examination procedure using a VTI probe may consist of eight tests which can be divided into three groups:
a. Low tissue deformation tests by moving a probe, when vaginal tissue is displaced from about 3 mm to about 15 mm—to characterize tissue elasticity:
   Probe insertion,
   Probe rotation,
b. High tissue deformation tests by moving a probe, when vaginal tissue is displaced from about 15 mm to about 45 mm—to characterize pelvic floor support structures:
   Probe elevation,
c. No tissue deformation tests (beyond initial probe insertion), when the probe is kept stationary and the patient is asked to perform actions causing voluntary and involuntary pelvic muscle contraction and relaxation—to characterize dynamic pelvic function:
   Valsalva maneuver,
   Voluntary muscle contraction, (anterior vs posterior),
   Voluntary muscle contraction (left side versus right side),
   Involuntary relaxation, and
   Reflex muscle contraction (resulting from a cough).
      Tests 1-5 and 7-8 provide data for anterior/posterior compartments; probe rotation as well as test 6 provides data for left/right sides (see Table 1).

TABLE 1
Exemplary VTI Examination includes 8 procedure tests.

| Test | | |
|---|---|---|
| No. | Procedure | Output |
| Test 1 | Probe | Tactile image for vaginal anterior and posterior |
| | insertion | compartments along the entire vagina |
| | | (resistance, force, work, tissue elasticity) |
| Test 2 | Probe | Tactile image for anterior and posterior |
| | elevation | compartments which related to pelvic floor |
| | | support structures (pressure value sand pressure |
| | | gradients for specified/critical locations) |
| Test 3 | Probe | Tactile images for left and right sides along |
| | rotation | the entire vagina (force and pressure values for |
| | | specified positions/locations) |
| Test 4 | Valsalva | Dynamic pressure response from opposite sites |
| | maneuver | (anterior vs posterior) along the entire vagina |
| | | (changes in force and pressure; pressure peak |
| | | displacements). |
| Test 5 | Voluntary | Dynamic pressure response from opposite sites |
| | muscle | (anterior vs posterior) along the entire vagina |
| | contraction | (changes in force and pressure; maximum |
| | | pressure values). |
| Test 6 | Voluntary | Dynamic pressure response from opposite sides |
| | muscle | (left vs right) along the entire vagina (changes |
| | contraction | in force and pressure; maximum pressure |
| | (sides) | values). |
| Test 7 | Involuntary | Dynamic pressure response from opposite sites |
| | relaxation | (anterior vs posterior) along the entire vagina |
| | | (changes in pressure). |
| Test 8 | Reflex muscle | Dynamic pressure response from opposite sites |
| | contraction | (anterior vs posterior) along the entire vagina |
| | (resulting | (changes in force and pressure; pressure peak |
| | from a cough) | displacements). |

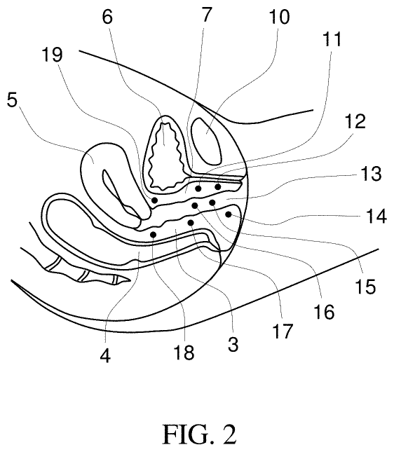

FIG. 2 shows exemplary locations of the tactile response parameters measured by a VTI probe for Tests 2 and 3 in mid-sagittal plane of the female pelvic floor. Specifically, location 12 captures anterior aspects of a perineal body opposite to a pubic bone 10; location 11 takes responses from a urethra (not shown); location 19 characterizes apical an anterior part connected to cervix of uterus 5; location 14 is related to Level III pelvic support; location 17 is related to Level II pelvic support; location 18 is related to Level I pelvic support; location 15 is related to vaginal site 1; and location 16 is related to vaginal side 2. Rectum 4 and bladder 6 are shown as anatomical landmarks.

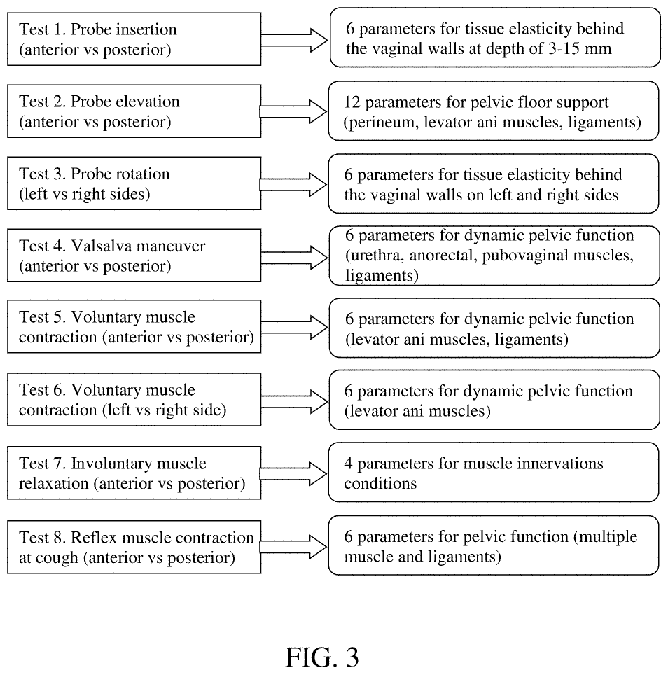

FIG. 3 shows a correlation of the Tests 1-8 with biomechanical parameters calculated using data obtained during these tests. Table 2 below provides further details and explanation of 52 exemplary biomechanical parameters derived from the VTI examination data.

TABLE 2
VTI Biomechanical Parameters.

| | | | | | | | Targeting/ |
|---|---|---|---|---|---|---|---|
| | | | | | | | Contributing |
| | VTI | Parameters | | Parameter | Parameter | Parameter | Pelvic |
| No. | Test | Abbreviation | Units | Description | Interpretation | Class | Structures |
| 1 | 1 | Fmax | N | Maximum value of force | Maximum resistance of | Maximum | Tissues |
| | | | | measured during the | anterior vs posterior | vaginal | behind the |
| | | | | VTI probe insertion | widening; tissue | tissue | anterior and |
| | | | | | elasticity at specified | elasticity at | posterior |
| | | | | | location (capability to | specified | vaginal walls |
| | | | | | resist to applied | location | at 3-15 mm |
| | | | | | deformation) | | depth |
| 2 | 1 | Work | mJ | Work completed during | Integral resistance of | Average | Tissues |
| | | | | probe insertion (Work = | vaginal tissue (anterior | vaginal | behind the |
| | | | | Force × Displacement) | and posterior) along | tissue | anterior and |
| | | | | | the probe insertion | elasticity | posterior |
| | | | | | | | vaginal walls |
| | | | | | | | at 3-15 mm |
| | | | | | | | depth |
| 3 | 1 | Gmax_a | kPa/mm | Maximum value of | Maximum value of | Maximum | Tissues/ |
| | | | | anterior gradient | tissue elasticity in | value of | structures |
| | | | | (change of pressure per | anterior compartment | anterior | in anterior |
| | | | | anterior wall | behind the vaginal at | tissue | compartment |
| | | | | displacement in | specified location | elasticity | at 10-15 mm |
| | | | | orthogonal direction to | | | depth |
| | | | | the vaginal channel) | | | |
| 4 | 1 | Gmax_p | kPa/mm | Maximum value of | Maximum value of | Maximum | Tissues/ |
| | | | | posterior gradient | tissue elasticity in | value of | structures |
| | | | | (change of pressure per | posterior compartment | posterior | in anterior |
| | | | | posterior wall | behind the vaginal at | tissue | compartment |
| | | | | displacement in | specified location | elasticity | at 10-15 mm |
| | | | | orthogonal direction to | | | depth |
| | | | | the vaginal channel) | | | |
| 5 | 1 | Pmax_a | kPa | Maximum value of | Maximum resistance of | Anterior | Tissues/ |
| | | | | pressure per anterior | anterior tissue to | tissue | structures |
| | | | | wall along the vagina | vaginal wall deformation | elasticity | in anterior |
| | | | | | | | compartment |
| 6 | 1 | Pmax_p | kPa | Maximum value of | Maximum resistance of | Posterior | Tissues/ |
| | | | | pressure per posterior | posterior tissue to | tissue | structures |
| | | | | wall along the vagina | vaginal wall deformation | elasticity | in posterior |
| | | | | | | | compartment |
| 7 | 2 | P1max_a | kPa | Maximum pressure at | Proximity of pubic bone | Anatomic | Tissues |
| | | | | the area of pubic bone | to vaginal wall and | aspects and | between |
| | | | | (anterior, 12 in FIG. 2) | perineal body strength | tissue | vagina and |
| | | | | | | elasticity | pubic bone; |
| | | | | | | | perineal body |
| 8 | 2 | P2max_a | kPa | Maximum pressure at | Elasticity/mobility of | Anatomic | Urethra and |
| | | | | the area of urethra | urethra | aspects and | surrounding |
| | | | | (anterior, 11 in FIG. 2) | | tissue | tissues |
| | | | | | | elasticity | |
| 9 | 2 | P3max_a | kPa | Maximum pressure at | Mobility of uterus and | Pelvic floor | Uterosacral |
| | | | | the cervix area (anterior, | conditions of | support | and cardinal |
| | | | | 19 in FIG. 2) | uterosacral and | | ligaments |
| | | | | | cardinal ligaments | | |
| 10 | 2 | P1max_p | kPa | Maximum pressure at | Pressure feedback of | Pelvic floor | Puboperineal, |
| | | | | the perineal body | Level III support | support | puborectal |
| | | | | (posterior, see 14 in | | | muscles |
| | | | | FIG. 2) | | | |
| 11 | 2 | P2max_p | kPa | Maximum pressure at | Pressure feedback of | Pelvic floor | Pubovaginal, |
| | | | | middle third of vagina | Level II support | support | puboanal |
| | | | | (posterior, see 17 in | | | muscles |
| | | | | FIG. 2) | | | |
| 12 | 2 | P3max_p | kPa | Maximum pressure at | Pressure feedback of | Pelvic floor | Iliococcygeal |
| | | | | upper third of vagina | Level I support | support | muscle, |
| | | | | (posterior, see 18 in | | | levator plate |
| | | | | FIG. 2) | | | |
| 13 | 2 | G1max_a | kPa/mm | Maximum gradient at | Vaginal elasticity at | Anterior | Tissues |
| | | | | the area of pubic bone | pubic bone area | tissue | between |
| | | | | (anterior, see 12 in FIG. 2) | | elasticity | vagina and |
| | | | | | | | pubic bone; |
| | | | | | | | perineal body |
| 14 | 2 | G2max_a | kPa/mm | Maximum gradient at | Mobility and elasticity | Urethral | Urethra and |
| | | | | the area of urethra | of urethra | tissue | surrounding |
| | | | | (anterior, see 11 in FIG. 2) | | elasticity | tissues |
| 15 | 2 | G3max_a | kPa/mm | Maximum gradient at | Conditions of | Pelvic floor | Uterosacral |
| | | | | the cervix area (anterior, | uterosacral and | support | and cardinal |
| | | | | see 19 in FIG. 2) | cardinal ligaments | | ligaments |
| 16 | 2 | G1max_p | kPa/mm | Maximum gradient at | Strength of Level III | Pelvic floor | Puboperineal, |
| | | | | the perineal body | support (tissue | support | puborectal |
| | | | | (posterior, see 14 in | deformation up to 25 mm) | | muscles |
| | | | | FIG. 2) | | | |
| 17 | 2 | G2max_p | kPa/mm | Maximum gradient at | Strength of Level II | Pelvic floor | Pubovaginal, |
| | | | | middle third of vagina | support (tissue | support | puboanal |
| | | | | (posterior, see 17 in | deformation up to 35 mm) | | muscles |
| | | | | FIG. 2) | | | |
| 18 | 2 | G3max_p | kPa/mm | Maximum gradient at | Strength of Level I | Pelvic floor | Iliococcygeal |
| | | | | upper third of vagina | support (tissue | support | muscle, |
| | | | | (posterior, see 18 in | deformation up to 45 mm) | | levator plate |
| | | | | FIG. 2) | | | |
| 19 | 3 | Pmax | kPa | Maximum pressure at | Hard tissue or tight | Vaginal | Tissues |
| | | | | vaginal walls | vagina | tissue | behind the |
| | | | | deformation by 7 mm | | elasticity | vaginal walls |
| | | | | | | | at 5-7 mm |
| | | | | | | | depth |
| 20 | 3 | Fap | N | Force applied by | Integral strength of | Vaginal | Tissues |
| | | | | anterior and posterior | anterior and posterior | tightening | behind |
| | | | | compartments to the | compartments | | anterior/ |
| | | | | probe | | | posterior |
| | | | | | | | vaginal walls. |
| 21 | 3 | Fs | N | Force applied by entire | Integral strength of left | Vaginal | Vaginal |
| | | | | left and right sides of | and right sides of | tightening | right/left walls |
| | | | | vagina to the probe | vagina | | and tissues |
| | | | | | | | behind them. |
| 22 | 3 | P1_l | kPa | Pressure response from | Hard tissue on left | Irregularity | Tissue/muscle |
| | | | | a selected location | vaginal wall | on vaginal | behind the |
| | | | | (irregularity 1) at left | | wall | vaginal walls |
| | | | | side (see 15 in FIG. 2) | | | on left side. |
| 23 | 3 | P2_l | kPa | Pressure response from | Hard tissue on left | Irregularity | Tissue/muscle |
| | | | | a selected location | vaginal wall | on vaginal | behind the |
| | | | | (irregularity 2) at left | | wall | vaginal walls |
| | | | | side (see 16 in FIG. 2) | | | on left side. |
| 24 | 3 | P3_r | kPa | Pressure response from | Hard tissue on right | Irregularity | Tissue/muscle |
| | | | | a selected location | vaginal wall | on vaginal | behind the |
| | | | | (irregularity 3) at right | | wall | vaginal walls |
| | | | | side (see 15 in FIG. 2) | | | on right side. |
| 25 | 4 | dF_a | N | Integral force change in | Pelvic function at | Pelvic | Multiple |
| | | | | anterior compartment at | Valsalva maneuver | function | pelvic |
| | | | | Valsalva maneuver | | | muscles |
| 26 | 4 | dPmax_a | kPa | Maximum pressure | Pelvic function at | Pelvic | Multiple |
| | | | | change in anterior | Valsalva maneuver | function | pelvic |
| | | | | compartment at | | | muscles |
| | | | | Valsalva maneuver. | | | |
| 27 | 4 | dL_a | mm | Displacement of the | Mobility of anterior | Pelvic | Urethra, |
| | | | | maximum pressure peak | structures, Valsalva | function | pubovaginal |
| | | | | in anterior compartment | maneuver | | muscle; |
| | | | | | | | ligaments |
| 28 | 4 | dF_p | N | Integral force change in | Pelvic function at | Pelvic | Multiple |
| | | | | posterior compartment | Valsalva maneuver | function | pelvic |
| | | | | at Valsalva maneuver | | | muscles |
| 29 | 4 | dPmax_p | kPa | Maximum pressure | Pelvic function at | Pelvic | Multiple |
| | | | | change in posterior | Valsalva maneuver | function | pelvic |
| | | | | compartment at | | | muscles |
| | | | | Valsalva maneuver. | | | |
| 30 | 4 | dL_p | mm | Displacement of the | Mobility of posterior | Pelvic | Anorectal, |
| | | | | maximum pressure peak | structures Valsalva | function | puborectal, |
| | | | | in posterior compartment | maneuver | | pubovaginal |
| | | | | | | | muscles; |
| | | | | | | | ligaments |
| 31 | 5 | dF_a | N | Integral force change in | Integral contraction | Pelvic | Puboperineal, |
| | | | | anterior compartment at | strength of pelvic | function | puborectal, |
| | | | | voluntary muscle | muscles along the | | pubovaginal |
| | | | | contraction | vagina | | and ilicoccygeal |
| | | | | | | | muscles; uretra |
| 32 | 5 | dPmax_a | kPa | Maximum pressure | Contraction strength of | Pelvic | Puboperineal, |
| | | | | change in anterior | specified pelvic | function | puborectal |
| | | | | compartment at | muscles | | and pubovaginal |
| | | | | voluntary muscle | | | muscles |
| | | | | contraction | | | |
| 33 | 5 | Pmax_a | kPa | Maximum pressure | Static and dynamic | Pelvic | Puboperineal |
| | | | | value in anterior | peak support of the | function | and puborectal |
| | | | | compartment at | pelvic floor | | muscles |
| | | | | voluntary muscle | | | |
| | | | | contraction. | | | |
| 34 | 5 | dF_p | N | Integral force change in | Integral contraction | Pelvic | Puboperineal, |
| | | | | posterior compartment | strength of pelvic | function | puborectal, |
| | | | | at voluntary muscle | muscles along the | | pubovaginal |
| | | | | contraction | vagina | | and ilicoccygeal |
| | | | | | | | muscles |
| 35 | 5 | dPmax_p | kPa | Maximum pressure | Contraction strength of | Pelvic | Puboperineal, |
| | | | | change in posterior | pelvic muscles at | function | puborectal |
| | | | | compartment at | specified location | | and pubovaginal |
| | | | | voluntary muscle | | | muscles |
| | | | | contraction | | | |
| 36 | 5 | Pmax_p | kPa | Maximum pressure | Static and dynamic | Pelvic | Puboperineal |
| | | | | value in posterior | peak support of the | function | and puborectal |
| | | | | compartment at | pelvic floor | | muscles |
| | | | | voluntary muscle | | | |
| | | | | contraction. | | | |
| 37 | 6 | dF_r | N | Integral force change in | Integral contraction | Pelvic | Puboperineal, |
| | | | | right side at voluntary | strength of pelvic | function | puborectal, |
| | | | | muscle contraction | muscles along the | | and pubovaginal |
| | | | | | vagina | | muscles |
| 38 | 6 | dPmax_r | kPa | Maximum pressure | Contraction strength of | Pelvic | Puboperineal |
| | | | | change in right side at | specific pelvic muscle | function | or puborectal |
| | | | | voluntary muscle | | | or pubovaginal |
| | | | | contraction | | | muscles |
| 39 | 6 | Pmaxa_r | kPa | Maximum pressure | Specified pelvic muscle | Pelvic | Puboperineal |
| | | | | value in right side at | contractive capability | function | or puborectal |
| | | | | voluntary muscle | and integrity | | muscles |
| | | | | contraction | | | |
| 40 | 6 | dF_l | N | Integral force change in | Integral contraction | Pelvic | Puboperineal, |
| | | | | left side at voluntary | strength of pelvic | function | puborectal, |
| | | | | muscle contraction | muscles along the | | and pubovaginal |
| | | | | | vagina | | muscles |
| 41 | 6 | dPmax_l | kPa | Maximum pressure | Contraction strength of | Pelvic | Puboperineal |
| | | | | change in left side at | specific pelvic muscle | function | or puborectal |
| | | | | voluntary muscle | | | or pubovaginal |
| | | | | contraction | | | muscles |
| 42 | 6 | Pmaxa_l | kPa | Maximum pressure | Specified pelvic muscle | Pelvic | Puboperineal |
| | | | | value in left side at | contractive capability | function | or puborectal |
| | | | | voluntary muscle | and integrity | | muscles |
| | | | | contraction | | | |
| 43 | 7 | dPdt_a | kPa/s | Anterior absolute | Innervation status of | Innervations | Levator ani |
| | | | | pressure change per | specified pelvic | status | muscles |
| | | | | second for maximum | muscles | | |
| | | | | pressure at involuntary | | | |
| | | | | relaxation | | | |
| 44 | 7 | dpcdt_a | %/s | Anterior relative | Innervation status of | Innervations | Levator ani |
| | | | | pressure change per | specified pelvic | status | muscles |
| | | | | second for maximum | muscles | | |
| | | | | pressure at involuntary | | | |
| | | | | relaxation | | | |
| 45 | 7 | dPdt_p | kPa/s | Posterior absolute | Innervation status of | Innervations | Levator ani |
| | | | | pressure change per | specified pelvic | status | muscles |
| | | | | second for maximum | muscles | | |
| | | | | pressure at involuntary | | | |
| | | | | relaxation | | | |
| 46 | 7 | dpcdt_p | %/s | Posterior relative | Innervation status of | Innervations | Levator ani |
| | | | | pressure change per | specified pelvic | status | muscles |
| | | | | second for maximum | muscles | | |
| | | | | pressure at involuntary | | | |
| | | | | relaxation | | | |
| 47 | 8 | dF_a | N | Integral force change in | Integral pelvic function | Pelvic | Multiple |
| | | | | anterior compartment at | at reflex muscle | function | pelvic muscle |
| | | | | reflex pelvic muscle | contraction | | |
| | | | | contraction (cough) | | | |
| 48 | 8 | dPmax_a | kPa | Maximum pressure | Contraction strength of | Pelvic | Multiple |
| | | | | change in anterior | specified pelvic | function | pelvic muscle |
| | | | | compartment at reflex | muscles | | |
| | | | | pelvic muscle | | | |
| | | | | contraction (cough). | | | |
| 49 | 8 | dL_a | mm | Displacement of the | Mobility of anterior | Pelvic | Urethra, |
| | | | | maximum pressure peak | structures at reflex | function | pubovaginal |
| | | | | in anterior compartment | muscle contraction | | muscle; ligaments |
| 50 | 8 | dF_p | N | Integral force change in | Integral pelvic function | Pelvic | Multiple |
| | | | | posterior compartment | at reflex muscle | function | pelvic muscle |
| | | | | at reflex pelvic muscle | contraction | | |
| | | | | contraction (cough) | | | |
| 51 | 8 | dPmax_p | kPa | Maximum pressure | Contraction strength of | Pelvic | Multiple |
| | | | | change in posterior | specified pelvic | function | pelvic muscle |
| | | | | compartment at reflex | muscles | | |
| | | | | pelvic muscle | | | |
| | | | | contraction (cough). | | | |
| 52 | 8 | dL_p | mm | Displacement of the | Mobility of anterior | Pelvic | Anorectal, |
| | | | | maximum pressure peak | structures at reflex | function | puborectal |
| | | | | in posterior compartment | muscle contraction | | and pubovaginal |
| | | | | | | | muscles; ligaments |

Capture of static tactile pattern may occur after 3-5 seconds following completion of probe insertion to allow vaginal tissue to get equilibrium in internal stress and strain distribution. To accurately record the static tactile pattern, probe 1 must be held in place without any displacements and keeping the probe oriented in parallel to vagina canal. The patient may be placed in a horizontal position during the probe insertion and capturing the static tactile pattern. Furthermore, the patient may be asked to contract vaginal muscles to enable recording of tactile signals on the rigid surface of the probe 1. The patient may be asked to follow the instruction from a medical professional as to the appropriate time for vaginal muscles contraction.

During Test 2, the probe can be tilted up and down (±20 degrees) by applying elevating (tilting) force relative to the hymen to record deeper transitional tactile response from median and apical anterior and posterior compartments. The recorded transitional tactile response provides vital information about biomechanical conditions of pelvic floor support structures. During Test 3, the probe may be rotated around its axis by 1 to 360 degrees to collect tactile data circumstantially from the vaginal walls. The recorded transitional tactile response provides vital information about biomechanical conditions of the vaginal walls and behind thereof at depths of 5-7 mm.

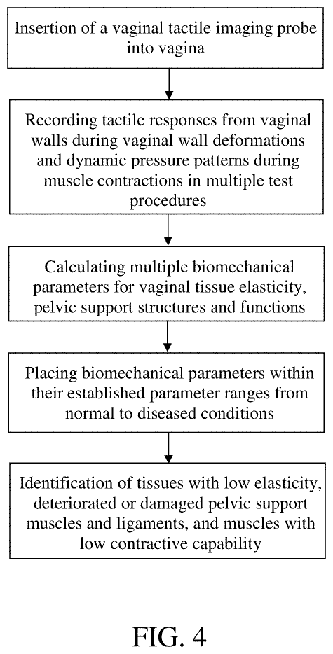

FIG. 4 illustrates one method for biomechanical mapping of the female pelvic floor comprising the steps of:
inserting a vaginal tactile imaging probe into vagina;
recording tactile responses for vaginal walls during vaginal wall deformation and dynamic pressure patterns during muscle contractions in multiple test procedures;
calculating multiple biomechanical parameters for vaginal tissue elasticity, pelvic support structures and functions;
placing every biomechanical parameter within the established parameter ranges from normal to diseased conditions; and
identification of tissues with low elasticity, deteriorated or damaged pelvic support muscles and ligaments, and muscles with low contractive capability.

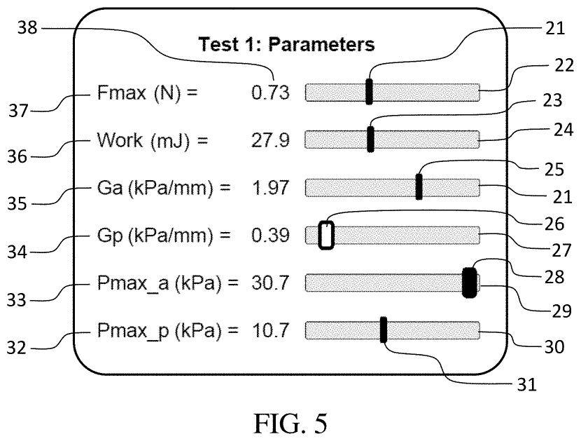

FIG. 5 shows an example of the visual presentation of the values of biomechanical parameters 32, 33, 34, 35, 36, 37 and their respective positions 31, 28, 26, 25, 23, 21 for the entire respective physiological ranges from minimum values on the left side to maximum values on the right side of the bars 30, 29, 27, 21, 24, 22. One, several or every parameter's numerical value may be presented by a number, e.g. for parameter 37, Fmax (N), the numerical number is 0.73 which is noted by 38. If the parameter falls in a lower 25% quartile, its appearance may be highlighted—for example to look like 26 (empty or white background); if the parameter falls in an upper 25% quartile, its appearance may also be highlighted to look like 28 (solid or black background).

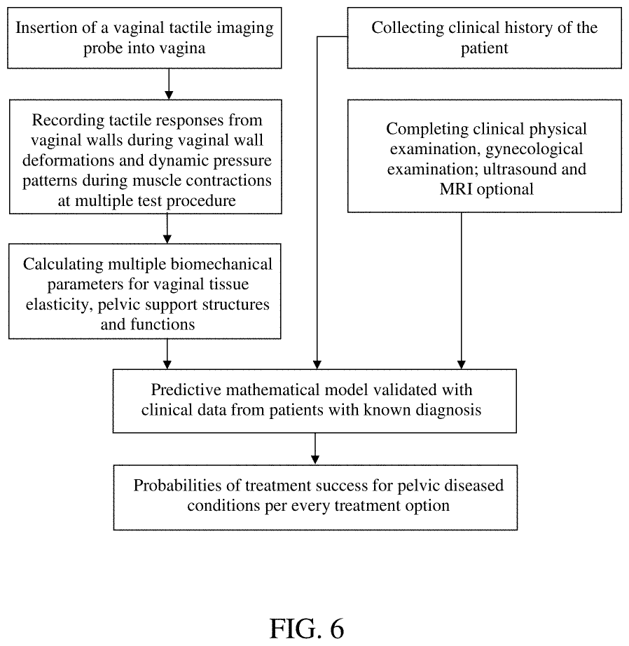

FIG. 6 illustrates another method for biomechanical mapping of the female pelvic floor comprising the steps of:
inserting a vaginal tactile imaging probe into vagina;
recording tactile responses for vaginal walls during vaginal wall deformation and dynamic pressure patterns during voluntary and involuntary muscle contractions in one or multiple test procedures;
calculating at least one or multiple biomechanical parameters characterizing one, two, or three of the following: vaginal tissue elasticity, pelvic support structures and dynamic pelvic functions;
collecting clinical history and completing gynecological examinations of the pelvic floor; and
calculating probabilities of treatment success for pelvic diseased conditions for individual proposed treatments.

A mathematical model of pelvic floor tissue deformations may be created and validated using clinical evaluation combined with calculating a plurality of parameters for biomechanical mapping for a sufficient number of subjects, including healthy subjects as well as a number of subjects with known anomalies of the pelvic floor structures. Once created and validated clinically, such mathematical model can be used prospectively to not only provide a more accurate diagnosis for a particular patient but also provide a prediction of clinical success in treatment of an anomaly of the pelvic floor tissues and structures.

In embodiments, a physical examination, ultrasound and/or magnetic resonance imaging (MRI) can be completed together with the gynecological examination to feed data into this mathematical model. Such predictive mathematical model may be based on an artificial intelligence algorithm and can be used to predict the probability of treatment success for a patient with pelvic floor diseased conditions. Diagnosis of a disease condition may also be conducted by comparing a measured value of one or more biomechanical parameters described above against their respective predetermined thresholds indicating a presence of such disease condition, which may be developed using clinical data from a plurality of patients.

Exemplary pelvic floor disease conditions may include pelvic organ prolapse, urinary or stress urinary incontinence, fecal incontinence, overactive bladder, vaginal tissue atrophy and/or pelvic pain. Exemplary treatments for such conditions can include a surgical treatment such as supracervical hysterectomy, total hysterectomy, saplingectomy, sacrocolpopexy, uterosacral ligament suspension, sling, anterior colporrhaphy, posterior colporrhaphy, enterocele repair and perineorrhaphy. Other contemplated treatments can include a non-surgical (conservative) treatment such as a physical therapy, weight loss, estrogen therapy, a pessary insertion, electrical neuromodulation, and/or a laser procedure.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method for biomechanical mapping of the female pelvic floor, said method comprising the steps of:
(a) inserting a vaginal tactile imaging probe into vagina, said probe equipped with a plurality of tactile sensors distributed along an external surface thereof;
(b) recording tactile response for vaginal walls in contact with said vaginal tactile imaging probe during vaginal wall deformation caused by moving said vaginal tactile imaging probe,
(c) recording dynamic pressure patterns for vaginal walls in contact with said vaginal tactile imaging probe during voluntary and involuntary muscle contractions and relaxations and without further movement of said vaginal tactile imaging probe;
(d) using said recorded tactile response in step (b) and said recorded dynamic pressure patterns in step (c) for calculating at least one biomechanical parameter to characterize each of:
i. vaginal tissue elasticity,
ii. pelvic support structures, and
iii. dynamic pelvic functions.

2. The method as in claim 1, wherein said step (b) further comprising moving said vaginal tactile imaging probe by about 3 to about 15 mm to cause low vaginal wall deformations, whereby said tactile response recorded during said low vaginal wall deformations is used to calculate at least one biomechanical parameter characterizing vaginal tissue elasticity.

3. The method as in claim 2, wherein said at least one biomechanical parameter characterizing vaginal tissue elasticity in step (d) is selected from a group consisting of:
maximum force measured during said vaginal tactile imaging probe insertion;
work completed during said vaginal tactile imaging probe insertion calculated as force multiplied by displacement;
maximum value of anterior gradient calculated as a change of pressure per anterior wall displacement in orthogonal direction to a vaginal canal;
maximum value of posterior gradient calculated as change of pressure per posterior wall displacement in orthogonal direction to said vaginal canal;
maximum value of pressure per anterior wall along said vaginal canal;
maximum value of pressure per posterior wall along said vaginal canal;
maximum pressure at vaginal walls deformation;
force applied by an anterior vaginal compartment and a posterior vaginal compartment to said vaginal tactile imaging probe;
force applied by entire left side of vagina and entire right side of vagina to said vaginal tactile imaging probe;
tactile pressure response from a selected location of irregularity on a left side of vagina; and
tactile pressure response from a selected location of irregularity on a right side of vagina.

4. The method as in claim 1, wherein said step (b) further comprising moving said vaginal tactile imaging probe by about 15 to about 45 mm to cause high vaginal wall deformations, whereby said tactile response recorded during said high vaginal wall deformations is used to calculate at least one biomechanical parameter characterizing pelvic support structures.

5. The method as in claim 4, wherein said at least one biomechanical parameter characterizing pelvic support structures in step (d) is selected from a group consisting of:
maximum pressure at a location of a pubic bone;
maximum pressure at a location of urethra;
maximum pressure at a cervix area;
maximum pressure at a perineal body;
maximum pressure at a middle third of vagina;
maximum pressure at an upper third of vagina;
maximum gradient at said location of the pubic bone;
maximum gradient at the area of urethra;
maximum gradient at the cervix area;
maximum gradient at the perineal body;
maximum gradient at said middle third of vagina; and
maximum gradient at said upper third of vagina.

6. The method as in claim 1, wherein said at least one biomechanical parameter characterizing dynamic pelvis function in step (d) is selected from a group consisting of:
integral force change in said anterior compartment at Valsalva maneuver;
maximum pressure change in said anterior compartment at Valsalva maneuver;
displacement of the maximum pressure peak in said anterior compartment;
integral force change in said posterior compartment at Valsalva maneuver;
maximum pressure change in said posterior compartment at Valsalva maneuver;
displacement of maximum pressure peak in said posterior compartment;
integral force change in said anterior compartment at voluntary muscle contraction;
maximum pressure change in said anterior compartment at voluntary muscle contraction;
maximum pressure value in said anterior compartment at voluntary muscle contraction;
integral force change in said posterior compartment at voluntary muscle contraction;
maximum pressure change in said posterior compartment at voluntary muscle contraction;
maximum pressure value in said posterior compartment at voluntary muscle contraction;
integral force change in the right side of vagina at voluntary muscle contraction;
maximum pressure change in the right side of vagina at voluntary muscle contraction;
maximum pressure in the right side of vagina at voluntary muscle contraction;
integral force change in the left side of vagina at voluntary muscle contraction;
maximum pressure change in the left side of vagina at voluntary muscle contraction;
maximum pressure value in the left side of vagina at voluntary muscle contraction;
anterior absolute pressure change per second for maximum pressure at involuntary relaxation;
anterior relative pressure change per second for maximum pressure at involuntary relaxation;
posterior absolute pressure change per second for maximum pressure at involuntary relaxation;
posterior relative pressure change per second for maximum pressure at involuntary relaxation;
integral force change in said anterior compartment at reflex pelvic muscle contraction caused by cough;
maximum pressure change in said anterior compartment at reflex pelvic muscle contraction caused by cough;
displacement of the maximum pressure peak in said anterior compartment;
integral force change in said posterior compartment at reflex pelvic muscle contraction caused by cough;
maximum pressure change in said posterior compartment at reflex pelvic muscle contraction caused by cough; and
displacement of the maximum pressure peak in said posterior compartment.

7. The method as in claim 1 further comprising a step of visually representing at least some of said biomechanical parameters within their respective physiologic parameter ranges varying from normal to disease condition.

8. The method as in claim 7 further comprising a step of identification of tissues with low elasticity, deteriorated or damaged pelvic support muscles and ligaments, and muscles with low contractive capability based on said values of biomechanical parameters calculated in step (d) and compared to their respective physiologic parameter ranges.

9. The method as in claim 1, wherein steps (b) and (c) are repeated multiple times and said biomechanical parameters are calculated in step (d) using data from multiple evaluations.

10. The method as in claim 1 further comprising a step establishing a diagnosis of a disease condition of a pelvic floor tissues based on comparing said calculated values for said biomechanical parameters against a predetermined threshold indicating said diseased condition.

11. The method as in claim 10, wherein said disease condition is selected from a group consisting of a pelvic organ prolapse, a urinary incontinence, a stress urinary incontinence, a fecal incontinence, an overactive bladder, a vaginal tissue atrophy, and a pelvic pain.

12. The method as in claim 1 further comprising a step of calculating a probability of success for a proposed treatment for said disease condition.

13. The method as in claim 12, wherein said step of calculating a probability of success for a proposed treatment is based on using a mathematical model of biomechanical properties of said pelvic floor tissues validated with clinical data collected from a plurality of patients with known diagnosis.

14. The method as in claim 13, wherein said mathematical model is further based on collected clinical history and completing gynecological examinations of the pelvic floor for said plurality of patients.

15. The method as in claim 14, wherein said step of completing gynecological examinations further comprises a physical examination and ultrasound imaging or magnetic resonance imaging.

16. The method as in claim 12, wherein said proposed treatment is selected from a group consisting of a supracervical hysterectomy, a total hysterectomy, a saplingectomy, a sacrocolpopexy, an uterosacral ligament suspension, implantation of a sling, an anterior colporrhaphy, a posterior colporrhaphy, an enterocele repair, a perineorrhaphy, a physical therapy, a weight loss, an estrogen therapy, a pessary insertion, an electrical neuromodulation, and a laser procedure.
